(19) United States
(12) Patent Application Publication
(10) Pub. No.: US 2021/0186895 A1
(43) Pub. Date: Jun. 24, 2021

(54) CEREBRAL HYPOFUNCTION INHIBITOR OR CEREBRAL HYPOFUNCTION PROPHYLACTIC AGENT CONTAINING CAROTENOID COMPOSITION

(71) Applicant: JXTG NIPPON OIL & ENERGY CORPORATION, Chiyoda-ku, Tokyo (JP)

(72) Inventors: Takashi ISHIBASHI, Tokyo (JP); Masahiro HAYASHI, Tokyo (JP)

(73) Assignee: JXTG NIPPON OIL & ENERGY CORPORATION, Chiyoda-ku, Tokyo (JP)

(21) Appl. No.: 16/756,199

(22) PCT Filed: May 24, 2018
(86) PCT No.: PCT/JP2018/020051

§ 371 Date: Apr. 15, 2020

(30) Foreign Application Priority Data
Oct. 27, 2017 (JP) 2017-208154

(51) Int. Cl. A61K 31/122; A23L 33/10; A23L 29/00

(52) U.S. Cl. CPC A61K 31/122; A23L 29/045; A23L 33/10

(57) ABSTRACT

The present invention relates to a prophylactic or inhibitory agent for brain hypofunction, which comprises a carotenoid composition or a carotenoid formulation, each containing astaxanthin, adonirubin and adonixanthin at a given ratio.

### TECHNICAL FIELD

The present invention relates to an inhibitory agent for brain hypofunction or a prophylactic agent for brain hypofunction, each containing a carotenoid composition.

### BACKGROUND ART

In recent years, our life expectancy has risen and our society has been aging due to improved living environment and advanced medical technology, etc. The rate of dementia increases with age, and approximately one in four people aged 85 years and over are deemed to have dementia. For this reason, in advanced nations having entered into aged society status, dementia has become a large social problem. One of the symptoms of dementia is a failure of memory, but such a failure of memory may also be caused by aging of the normal brain. Namely, dementia is a disease in the brain, whereas aging-induced brain hypofunction such as reduced memory ability or reduced information processing ability is one of the aging phenomena which may occur in anyone.

On the other hand, carotenoids including astaxanthin are known to have various effects. For example, astaxanthin is known to have an antioxidant effect to scavenge active oxygen species and a prophylactic effect on retinopathy (Patent Document 1). However, carotenoids such as adonirubin and adonixanthin are not known to have an inhibitory or prophylactic effect on reduction in the brain function such as verbal memory ability or information processing ability. The above carotenoids have chemical structures similar to each other, but differ in their effect depending on their inherent structure; and hence their function cannot be inferred from their structural similarity. For example, zeaxanthin and lutein each have a structure similar to that of astaxanthin, but zeaxanthin and lutein are accumulated exclusively around the macula lutea in eyes, whereas astaxanthin reaches blood vessels in eyes but is not accumulated therein. Thus, various carotenoids exert different effects in different sites depending on their binding protein; and hence their effect cannot be predicted solely from their structural similarity. Namely, when attempting to identify the function of a certain carotenoid, some tests required for this purpose should be performed on this carotenoid. Carotenoids are obtained by being chemically synthesized or by being extracted from naturally occurring products of animal, plant, microorganism or other origin. In terms of being applied to human use, carotenoids are desired to be extracted from naturally occurring products for safety reasons.

### PRIOR ART DOCUMENTS

### Patent Documents

Patent Document 1: JP 2015-140346 A

### SUMMARY OF THE INVENTION

### Problem to be Solved by the Invention

In view of these circumstances, the present invention aims to provide a prophylactic or inhibitory agent for brain hypofunction such as reduced verbal memory ability or reduced information processing ability, which is safe for human use.

### Means to Solve the Problem

As a result of extensive and intensive efforts made to solve the problems stated above, the inventors of the present invention have found that a carotenoid composition containing astaxanthin, adonirubin and adonixanthin at a given ratio has a prophylactic or inhibitory effect on reduction in the brain function such as verbal memory ability or information processing ability. This finding led to the completion of the present invention.

Namely, the present invention encompasses the following embodiments.

[1] An inhibitory or prophylactic agent for brain hypofunction, which comprises a carotenoid composition containing astaxanthin, adonirubin and adonixanthin.
[2] The agent according to [1] above, wherein the carotenoid composition contains 45% to 80% by mass of astaxanthin, 4% to 22% by mass of adonirubin and 4% to 20% by mass of adonixanthin, on the basis of the total mass of the composition.
[3] An inhibitory or prophylactic agent for brain hypofunction, which comprises a carotenoid formulation containing astaxanthin, adonirubin and adonixanthin.
[4] The agent according to [3] above, wherein the carotenoid formulation contains 0.5% to 20% by mass of astaxanthin, 0.01% to 4% by mass of adonirubin and 0.01% to 4% by mass of adonixanthin, on the basis of the total mass of the formulation.
[5] The agent according to any one of [1] to [4] above, characterized in that the astaxanthin has no ester structure.
[6] The agent according to any one of [1] to [5] above, characterized in that the adonirubin has no ester structure.
[7] The agent according to any one of [1] to [6] above, characterized in that the adonixanthin has no ester structure.
[8] The agent according to any one of [1] to [7] above, wherein the brain function is verbal memory ability.
[9] The agent according to any one of [1] to [7] above, wherein the brain function is information processing ability.
[10] The agent according to any one of [1] to [7] above, wherein the brain hypofunction is aging-induced brain hypofunction.
[11] A pharmaceutical composition, a food composition or a drinkable composition, which contains the agent according to any one of [1] to [10] above.
[12] A method for inhibiting or preventing brain hypofunction, which comprises administering a carotenoid composition or a carotenoid formulation, each containing astaxanthin, adonirubin and adonixanthin, to a subject.
[13] The method according to [12] above, wherein the carotenoid composition contains 45% to 80% by mass of astaxanthin, 4% to 22% by mass of adonirubin and 4% to 20% by mass of adonixanthin, on the basis of the total mass of the composition.
[14] The method according to [12] above, wherein the carotenoid formulation contains 0.5% to 20% by mass of astaxanthin, 0.01% to 4% by mass of adonirubin and 0.01% to 4% by mass of adonixanthin, on the basis of the total mass of the formulation.
[15] The method according to any one of [12] to [14] above, wherein the brain function is verbal memory ability or information processing ability.
[16] The method according to any one of [12] to [14] above, wherein the brain hypofunction is aging-induced brain hypofunction.

### Effects of the Invention

The present invention provides a prophylactic agent for brain hypofunction or an inhibitory agent for brain hypofunction. Moreover, in a preferred embodiment, the present invention provides a prophylactic agent for aging-induced brain hypofunction or an inhibitory agent for aging-induced brain hypofunction.

### BRIEF DESCRIPTION OF THE DRAWINGS

FIG. 1 shows the results of verbal memory test in Inventive Example 1 and Comparative Example 1.

### DESCRIPTION OF EMBODIMENTS

The present invention will be described in more detail below.

The present invention relates to a prophylactic or inhibitory agent for brain hypofunction such as reduced verbal memory ability or reduced information processing ability, which comprises a carotenoid composition containing astaxanthin, adonirubin and adonixanthin at a given ratio. Moreover, the present invention relates to a prophylactic or inhibitory agent for brain hypofunction such as reduced verbal memory ability or reduced information processing ability, which comprises a carotenoid formulation containing astaxanthin, adonirubin and adonixanthin at a given ratio.

The present invention is based on the finding that a carotenoid composition or formulation containing astaxanthin, adonirubin and adonixanthin at a given ratio has a prophylactic or inhibitory effect on reduction in the brain function such as verbal memory ability or information processing ability.

In more detail, the inventors of the present invention have made the following study: healthy middle-aged and elderly male and female subjects aged 45 to 64 years were each allowed to take a food product containing carotenoids such as astaxanthin, adonirubin and adonixanthin (hereinafter referred to as a carotenoid-containing food product) for 4 weeks or longer, and the effects of the carotenoid-containing food product on their brain function were examined by word memory test, word recall test and Stroop test where a placebo was used as a control. Each test was performed as a randomized, double-blind, placebo-controlled, parallel-group comparison. Those with a history of cranial nerve disease and those suspected to have dementia because of their low scores on the dementia scale were excluded from the subjects.

As a result, for example, in 3 items of the word memory test performed on subjects aged less than 55 years, their amounts of change at 4 weeks were significantly increased in the subject group receiving the carotenoid-containing food product when compared to the placebo group. Likewise, serum carotenoids (astaxanthin, adonirubin, adonixanthin) were all significantly increased in the group receiving the carotenoid-containing food product when compared to the placebo group.

These results indicated that among middle-aged and elderly subjects, male and female subjects aged 45 to 54 years were found to improve or enhance their verbal memory ability when allowed to take the carotenoid composition for 4 weeks or longer.

Moreover, for example, in subjects aged 55 years and over, their information processing ability was found to be enhanced.

Thus, a carotenoid composition or formulation comprising astaxanthin, adonirubin, adonixanthin and others can be used as a prophylactic or inhibitory agent for brain hypofunction. When administered to subjects (e.g., human), the prophylactic or inhibitory agent for brain hypofunction according to the present invention will be able to prevent or inhibit brain hypofunction, e.g., reduced verbal memory ability or reduced information processing ability.

Moreover, in the above tests, those with a history of cranial nerve disease and those suspected to have dementia because of their low scores on the dementia scale were excluded from the subjects; and hence examination was made of the effect on aging-induced brain hypofunction, but not on brain hypofunction resulting from dementia or other diseases. Thus, in another embodiment of the present invention, the carotenoid composition or formulation of the present invention is regarded as being particularly effective in the inhibition or prevention of aging-induced brain hypofunction.

Further, the carotenoid composition or formulation of the present invention was found to have an improving effect on verbal memory ability in subjects aged less than 55 years; and hence in yet another embodiment of the present invention, the carotenoid composition or formulation of the present invention is regarded as being particularly effective in the inhibition or prevention of brain hypofunction in the early stage of aging. Likewise, the carotenoid composition or formulation of the present invention was found to have an improving effect on information processing ability in subjects aged 55 years and over; and hence in yet another embodiment of the present invention, the carotenoid composition or formulation of the present invention is also regarded as being particularly effective in the inhibition or prevention of brain hypofunction in and after the early stages of aging.

The carotenoid composition or formulation of the present invention comprises astaxanthin, adonirubin and adonixanthin. In addition to astaxanthin, adonirubin and adonixanthin, the carotenoid composition or formulation of the present invention may further contain one or more carotenoids selected from the group consisting of β-carotene, echinenone, 3-hydroxyechinenone, canthaxanthin and asteroidenone.

Carotenoids may be in monoester or diester form when their terminal hydroxy group(s) is esterified with a saturated fatty acid or an unsaturated fatty acid, etc. Carotenoids in the present invention (e.g., astaxanthin, adonirubin or adonixanthin) are more preferably those having no ester structure. This is because carotenoids in ester form will be deesterified during their transfer into blood, whereas carotenoids having no ester structure will not undergo desertification and are therefore highly absorbable. Carotenoids produced by microorganisms belonging to the genus Paracoccus have no ester structure.

The carotenoid composition of the present invention comprises:

astaxanthin in an amount whose lower limit is preferably 45% by mass or more, more preferably 50% by mass or more or 55% by mass or more, most preferably 60% by mass or more or 65% by mass or more, and whose upper limit is preferably 80% by mass or less, more preferably 75% by mass or less, most preferably 73% by mass or less, on the basis of the total mass (100%) of the composition;

adonirubin in an amount whose lower limit is preferably 4% by mass or more, more preferably 6% by mass or more, most preferably 8% by mass or more, and whose upper limit is preferably 22% by mass or less, more preferably 18% by mass or less, most preferably 15% by mass or less, on the basis of the total mass (100%) of the composition; and

adonixanthin in an amount whose lower limit is preferably 4% by mass or more, more preferably 7% by mass or more, most preferably 10% by mass or more, and whose upper limit is preferably 20% by mass or less, more preferably 15% by mass or less, most preferably 13% by mass or less, on the basis of the total mass (100%) of the composition.

These lower and upper limits may be combined in any combination. For example, the carotenoid composition of the present invention contains 45% to 80% by mass of astaxanthin, 4% to 22% by mass of adonirubin and 4% to 20% by mass of adonixanthin, on the basis of the total mass of the composition.

The carotenoid composition of the present invention may be a commercially available product or may be prepared by conventional chemical synthesis techniques, or alternatively, may be prepared by mixing chemically synthesized or naturally occurring carotenoids within the compositional range mentioned above. For human use, naturally occurring carotenoids are more preferred in terms of safety. The carotenoid composition of the present invention may comprise naturally occurring carotenoids produced by microbial fermentation or by extraction and purification from animals, plants, etc. For example, the carotenoid composition of the present invention may comprise carotenoids produced by being extracted and purified from microorganisms belonging to the genus Paracoccus, as exemplified by Paracoccus carotinifaciens, etc.

The carotenoid composition of the present invention may be prepared in accordance with the procedures described in JP 2009-50237 A. More specifically, a dried product of a microorganism belonging to the genus Paracoccus is extracted with ethanol at 90° C. to 100° C. for 15 minutes or longer to prepare a carotenoid eluate. The carotenoid eluate is filtered under heating (60° C. to 70° C.) to remove microbial cell debris. The filtrate (extract) is cooled to 30° C. and concentrated to 1/5 volume while adjusting the degree of vacuum to keep the vessel temperature at 30° C. After concentration, the filtrate is heated at 60° C. for 2 to 4 hours and further matured at 30° C. for 10 to 20 hours. After maturing, a carotenoid composition containing astaxanthin, adonixanthin and adonirubin is collected by filtration and then heated to dryness at 100° C. under vacuum. In this way, a carotenoid composition extracted from a microorganism belonging to the genus Paracoccus (e.g., Paracoccus carotinifaciens) may be obtained by the preparation procedures described later in the Example section.

The carotenoid composition described above can be used to prepare the inhibitory or prophylactic agent for brain hypofunction according to the present invention.

In the inhibitory or prophylactic agent for brain hypofunction according to the present invention, the carotenoid composition may be used directly, but it is also possible to use a formulation (carotenoid formulation) containing the carotenoid composition together with carriers acceptable for use in pharmaceuticals, carriers acceptable for use in food products, or carriers acceptable for use in beverages, etc.

The carotenoid formulation of the present invention comprises:

astaxanthin in an amount whose lower limit is preferably 0.5% by mass or more, more preferably 0.7% by mass or more, most preferably 0.9% by mass or more, and whose upper limit is preferably 20% by mass or less or 10% by mass or less, more preferably 5% by mass or less, most preferably 3% by mass or less, on the basis of the total mass (100%) of the formulation;

adonirubin in an amount whose lower limit is preferably 0.01% by mass or more or 0.05% by mass or more, more preferably 0.10% by mass or more, most preferably 0.12% by mass or more, and whose upper limit is preferably 4% by mass or less, more preferably 1% by mass or less, most preferably 0.5% by mass or less or 0.2% by mass or less, on the basis of the total mass (100%) of the formulation; and

adonixanthin in an amount whose lower limit is preferably 0.01% by mass or more, more preferably 0.03% by mass or more, most preferably 0.04% by mass or more, and whose upper limit is preferably 4% by mass or less, more preferably 1% by mass or less or 0.5% by mass or less, most preferably 0.2% by mass or less or 0.1% by mass or less, on the basis of the total mass (100%) of the formulation.

These lower and upper limits may be combined in any combination. For example, the carotenoid formulation of the present invention contains 0.5% to 20% by mass of astaxanthin, 0.01% to 4% by mass of adonirubin and 0.01% to 4% by mass of adonixanthin, on the basis of the total mass of the formulation.

Carriers for use in the present invention may be selected as appropriate from excipients, diluents, binders, extenders, lubricants, disintegrants, stabilizers, wetting agents, emulsifiers, buffering agents, suspending agents, preservatives or antioxidants, pH adjusters, gelling agents, solubilizers, coloring agents, flavoring agents and sweeteners, etc., and those commonly used for formulation purposes may be used in the preparation of the inhibitory or prophylactic agent for brain hypofunction according to the present invention. Examples include starches, sugars (e.g., dextrose, lactose, sucrose, glucose), sugar alcohols (e.g., mannitol, xylitol, erythritol, sorbitol, maltitol), acacia gum, gum arabic, gelatin, inorganic compounds (e.g., magnesium aluminometasilicate, hydrotalcite, anhydrous calcium phosphate, calcium carbonate, calcium silicate, light anhydrous silicic acid), polyvinylpyrrolidone, hydroxypropyl methylcellulose, microcrystalline cellulose, talc, silica, magnesium stearate, sodium starch glycolate, sodium lauryl sulfate, cellulose derivatives, methyl-p-hydroxybenzoate, sorbic acid, water, mineral oils and so on. However, carriers available for use in the present invention are not limited to these examples, and may also be exemplified by gum arabic, maltodextrin, tocopherol, medium-chain fatty acids and ascorbic acid.

The inhibitory or prophylactic agent for brain hypofunction according to the present invention may be in any dosage form. For use as a pharmaceutical composition, the dosage form may be designed for oral administration or parenteral administration (e.g., intravenous, intraarterial, intraperitoneal, transrectal, subcutaneous, intramuscular, sublingual, transnasal, transvaginal) by way of example. Such a dosage form is not limited in any way, and examples include solutions, tablets, oral rapidly disintegrating tablets, powders, granules, capsules, syrups, injections, suppositories, sprays, ointments, cataplasms, drinkable preparations and so on.

The inhibitory or prophylactic agent for brain hypofunction according to the present invention may be added to or filled into any form such as tablets, capsules, granules, tablets, jellies, gummies, gums, drinks, plastic bottles, etc., or alternatively, may be added to any food or beverage products substantially free from carotenoids, whereby the inhibitory or prophylactic agent for brain hypofunction according to the present invention may be provided as a food composition or a drinkable composition. As to the form of such a food composition or drinkable composition, examples include, but are not limited to, functional foods, health foods, supplements, confectioneries (jelly, yogurt, pudding, biscuits or cookies, chocolate, candy, cake, ice cream, chewing gum), ready-to-eat foods, soups, juices, tea products, jelly beverages, powder beverages, dairy products and so on. Moreover, such a food composition or drinkable composition may optionally comprise sweeteners, flavor enhancers, emulsifiers, suspending agents, antiseptics, etc. Further, the food composition or drinkable composition of the present invention may also be used as a food additive.

The dose of the carotenoid composition or formulation of the present invention used to inhibit or prevent brain hypofunction will vary depending on the patient's age, body weight, sex, condition and other factors. For example, the daily dose of the inhibitory or prophylactic agent for brain hypofunction according to the present invention is 2 to 24 mg/day, preferably 4 to 12 mg/day, when calculated as astaxanthin, but is not limited to this range. When required, the above dose may be administered once or divided into several doses (e.g., 2 to 3 doses) per day. Moreover, the inhibitory or prophylactic agent for brain hypofunction according to the present invention may be administered to a subject in combination with other inhibitory or prophylactic agents for brain hypofunction. Calculation as astaxanthin means that the dose of carotenoids contained in the carotenoid composition or formulation is representatively expressed as the amount of astaxanthin contained therein.

Moreover, the present invention also relates to a method for preventing or inhibiting brain hypofunction, which comprises administering a carotenoid composition or formulation to a patient. In more detail, the method for preventing or inhibiting brain hypofunction is in accordance with the descriptions about the inhibitory or prophylactic agent for brain hypofunction according to the present invention.

The brain function intended in the present invention includes verbal memory ability or information processing ability. Thus, the inhibitory or prophylactic agent for brain hypofunction according to the present invention can also be used to inhibit or prevent reduction in the verbal memory ability or to inhibit or prevent reduction in the information processing ability.

Moreover, brain hypofunction to be inhibited or prevented in the present invention may be aging-induced brain hypofunction. The subjects tested in the Example section are middle-aged and elderly subjects excluding those with a history of cranial nerve disease and those suspected to have dementia because of their low scores on the dementia scale. Thus, the inhibitory or prophylactic agent for brain hypofunction according to the present invention may be effective in the inhibition or prevention of aging-induced brain hypofunction.

The inhibitory or prophylactic agent for brain hypofunction according to the present invention can be assessed for its efficacy, for example, by word memory test, word recall test or Stroop test, as described later in the Example section. For example, after the inhibitory or prophylactic agent for brain hypofunction according to the present invention is administered to subjects, if the resulting test results are improved or enhanced in comparison with the test results obtained before administration or the control test results obtained upon placebo administration, the inhibitory or prophylactic agent for brain hypofunction according to the present invention can be determined to have an inhibitory or prophylactic effect on brain hypofunction. Such inhibitory and prophylactic effects on brain hypofunction also include improvement of the brain function, maintenance of the brain function, reduction in the severity of brain hypofunction, and reduction in the pace of brain hypofunction.

Verbal memory ability is the ability to memorize language items, and it has been known that the ability to memorize language items includes short-term and long-term abilities. In general, the word memory test is considered to be a test for confirming the short-term verbal memory ability, while the word recall test is considered to be a test for confirming the long-term verbal memory ability. Thus, a prophylactic or inhibitory effect on reduction in the verbal memory ability can be confirmed by the word memory test or the word recall test, as tested in the Example section.

Information processing ability is the brain's ability to process information, and will affect the amount of information which can be processed by the brain and/or the speed of information processing by the brain, etc. In general, the Stroop test is known to be a test for confirming selective attention, the speed of information processing, etc. Thus, a prophylactic or inhibitory effect on reduction in the information processing ability can be confirmed by the Stroop test, as tested in the Example section.

It should be noted that all documents and publications cited herein are incorporated herein by reference in their entirety, regardless of their purposes. Moreover, this specification incorporates the contents disclosed in the claims, specification and drawings of Japanese Patent Application No. 2017-208154 (filed on Oct. 27, 2017), based on which the present application claims priority.

### EXAMPLES

The present invention will be further described in more detail by way of the following illustrative examples, although the present invention is not limited only to these examples.

[Test Procedures]

### 1. Inclusion and Exclusion Criteria for Subjects

The subjects intended are male and female subjects aged 45 to 64 years at the time of giving informed consent, who do not fall within the following exclusion criteria. Moreover, these subjects excluding those falling within the following analysis exclusion criteria were provided for analysis.

Exclusion Criteria

(1) Subjects who are difficult to discriminate color

(2) Subjects who have a score of 20 or less on the Revised Hasegawa Dementia Scale

(3) Subjects with a history of cranial nerve disease

(4) Subjects with depressive symptoms or with a history thereof

(5) Subjects who are under treatment of their brain function, sleep or stress, and subjects who are prescribed with medicaments related thereto

Analysis Exclusion Criteria

(1) Subjects whose rate of carotenoid-containing food product intake is below 80%

(2) Subjects who are markedly observed to do actions impairing the reliability of the test results such as loss of records in their diaries

(3) Subjects who were found to have fallen within the exclusion criteria after enrollment in the test, and subjects who were found to be unable to maintain compliance with the restrictions during the test period

(4) Other subjects who have a clear reason for believing that they should be excluded

### 2. Background Factors of Subjects

The background factors of subject groups aged less than 55 years (45 to 54 years) and aged 55 years and over (55 to 64 years) are shown in Table 1 and Table 2, respectively. Except for their age, there were no large differences in their background factors between the subject group aged less than 55 years and the subject group aged 55 years and over.

TABLE 1
Background factors of the subject group aged less than 55 years

| | | Comparative group | Inventive group | |
|---|---|---|---|---|
| Item | Unit | (n = 14) | (n = 11) | p-value |
| Age | years | 49.7 ± 3.1 | 50.5 ± 2.7 | 0.537 |
| Sex (male/female) | number | male: 6/female: 8 | male: 3/female: 8 | 0.677 |
| Body height | cm | 161.98 ± 9.06 | 161.01 ± 6.52 | 0.768 |
| Body weight | kg | 57.64 ± 12.12 | 55.35 ± 6.39 | 0.577 |
| BMI | kg/m² | 21.80 ± 3.09 | 21.32 ± 1.87 | 0.657 |
| Systolic blood pressure | mmHg | 114.0 ± 16.0 | 111.6 ± 14.5 | 0.706 |
| Diastolic blood pressure | mmHg | 68.4 ± 11.5 | 68.4 ± 11.6 | 0.999 |
| Pulse rate | bpm | 68.1 ± 6.2 | 65.6 ± 9.1 | 0.421 |
| HDS-R score | points | 28.9 ± 1.0 | 29.2 ± 0.8 | 0.389 |

The data is expressed as the number of subjects for sex and expressed as mean ± standard deviation for the other items.
Inter-group comparison with the comparative group (sex: χ² test, items other than sex: two-sample t-test)

TABLE 2
Background factors of the subject group aged 55 years and over

| | | Comparative group | Inventive group | |
|---|---|---|---|---|
| Item | Unit | (n = 12) | (n = 17) | p-value |
| Age | years | 59.9 ± 3.1 | 59.6 ± 2.6 | 0.759 |
| Sex (male/female) | number | male: 7/female: 5 | male: 9/female: 8 | 1.000 |
| Body height | cm | 166.03 ± 8.90 | 163.26 ± 10.69 | 0.470 |
| Body weight | kg | 62.04 ± 14.34 | 65.17 ± 13.88 | 0.560 |
| BMI | kg/m² | 22.23 ± 3.22 | 24.23 ± 3.15 | 0.106 |
| Systolic blood pressure | mmHg | 134.2 ± 13.6 | 132.1 ± 16.5 | 0.727 |
| Diastolic blood pressure | mmHg | 81.7 ± 11.4 | 77.8 ± 10.6 | 0.352 |
| Pulse rate | bpm | 74.7 ± 10.4 | 70.1 ± 10.6 | 0.255 |
| HDS-R score | points | 28.3 ± 1.4 | 28.1 ± 2.7 | 0.753 |

The data is expressed as the number of subjects for sex and expressed as mean ± standard deviation for the other items.
Inter-group comparison with the comparative group (sex: χ² test, items other than sex: two-sample t-test)

### 3. Procedures for Brain Function Tests

In this experiment, the brain function was assessed using tests designed to assess memory ability and recall ability (word memory test and word recall test) and Stroop test designed to assess information processing ability and attention function. Moreover, serum carotenoid (astaxanthin, adonirubin, adonixanthin) levels were measured, thus confirming that carotenoids were absorbed after intake of a carotenoid-containing food product.

### 4. Word Memory Test (Confirmation Test for Verbal Memory Ability)

This “word memory test” is designed to assess the capacity of immediate and short-term memory as well as and ability to retain and repeat the memory. This test was performed by reference to the test method of Imamura (Yoko Imamura: Manual for the Clinical Assessment of Higher Brain Function 2000, revised 2nd edition, Shinkoh Igaku Shuppansha Co., Ltd., Japan, 2001).

In more detail, after reading seven words aloud one at a time to the subjects, they were asked to repeat the words. Immediately after they repeated the seventh word, they were asked to recall all of the words (the “number of immediate free recall words”). The subjects were given hints consisting of the initial sounds and categories of the words when they were unable to spontaneously recall these words. The words which the subjects were then able to recall were recorded as the “number of cued recall words.” Subsequently, the subjects were given interference tasks (word recall test) and were then asked to recall the words 5 minutes after the immediate free recall test (the “number of words freely recalled after 5 min”). Once again, the subjects were given hints consisting of the initial sounds and categories of the words when they were unable to spontaneously recall these words. The number of cued recall words which the subjects were then able to recall was recorded as the “number of words freely recalled after 5 min+cued recall words.” In this way, in the word memory test, the subjects were assessed in 4 stages, i.e., (1) the number of immediate free recall words, (2) the number of immediate free recall words+cued recall words, (3) the number of words freely recalled after 5 min, and (4) the number of words freely recalled after 5 min+cued recall words. In the word memory test, increases in the measured values indicate improved or enhanced verbal memory ability.

### 5. Word Recall Test (Confirmation Test for Verbal Memory Ability)

This “word recall test” is designed to assess the ability to recall memory. This test was performed by reference to the test method of Imamura (Yoko Imamura: Manual for the Clinical Assessment of Higher Brain Function 2000, revised 2nd edition, Shinkoh Igaku Shuppansha Co., Ltd., Japan, 2001).

For example, in a “vegetable” recall test, subjects were instructed to “say the names of vegetables to the greatest extent possible for 1 minute.” After the name of a first candidate was mentioned, words which the subjects were able to recall within 60 seconds were written down. The number of words which the subjects were able to correctly recall, excluding duplicates, was recorded. The same procedure was repeated to perform recall tests for “words beginning with “a”” and “animal names.” These tests were performed in differing order before intake and at 4 and 8 weeks after intake. The number of words which the subjects were able to correctly recall in each test was assessed. In the word recall test, increases in the measured values indicate improved or enhanced verbal memory ability.

### 6. Stroop Test (Confirmation Test for Information Processing Ability)

This “Stroop test” is designed to assess the ability to distinguish and process two different types of information, i.e., verbal information and color information that enter the brain simultaneously. This test was performed in accordance with the testing procedures of New Stroop Test II (Yuji Hakoda, Megumi Watanabe, New Stroop Test II, Toyo Physical Co., Ltd., Japan, http://www.toyophysical.co.jp/sinnsutoru-pul.htm).

The Stroop test consists of 4 steps, i.e., “Step 1,” “Step 2,” “Step 3” and “Step 4,” which are separate subtests, and the level of difficulty increases as the steps advance. The subjects were assessed for the total number of answers (the number of correct answers+the number of wrong answers), the number of correct answers and the number of wrong answers in each step. Increases in the measured values for the total number of answers and the number of correct answers indicate improved or enhanced information processing ability. Likewise, decreases in the measured values for the number of wrong answers indicate improved or enhanced information processing ability.

| | |
|---|---|
| Step 1 | Place a check mark in the check box for the color of |
| | ink indicated by a word. |
| Step 2 | Place a check mark in the check box for the color of |
| | ink indicated by a word which does not match the |
| | color of the ink. |
| Step 3 | Select a word corresponding to the color of ink, |
| | and place a check mark in its check box. |
| Step 4 | Select a word corresponding to the color of ink |
| | used to write the word which does not match the |
| | color of the ink, and place a check mark in |
| | its check box. |

### 7. Assessment of Efficacy

The amount of change at 4 or 8 weeks after intake in comparison with before intake was compared between the placebo group and the carotenoid-containing food product group by two-sample t-test. Moreover, in each group, the amount of change at each time point after intake in comparison with before intake was assessed by one-sample t-test.

The data was expressed as mean±standard deviation, and the significance level of the test was set to 5% on both sides. For inter-group comparison, 10% on both sides was regarded as marginal significance, which was also shown.

[Preparation of a Carotenoid Formulation, a Carotenoid-Containing Food Product and a Placebo]

In accordance with the procedures described in JP 2009-50237 A, a dried product of a microorganism belonging to the genus Paracoccus was extracted with ethanol at 90° C. for 20 minutes to prepare a carotenoid eluate. The carotenoid eluate was filtered under heating at 65° C. to remove microbial cell debris. The filtrate (extract) was cooled to 30° C. and concentrated to 1/5 volume while adjusting the degree of vacuum to keep the vessel temperature at 30° C. After concentration, the filtrate was heated at 60° C. for 3 hours and further matured at 30° C. for 12 hours. After maturing, a carotenoid composition containing astaxanthin, adonixanthin and adonirubin was collected by filtration and then heated to dryness at 100° C. under vacuum. The composition of the resulting carotenoid composition is shown in Table 3.

TABLE 3
Composition of the carotenoid composition

| | Relative proportion |
|---|---|
| Ingredient | (% by mass) |
| Astaxanthin | 69.9 |
| Adonirubin | 9.8 |
| Adonixanthin | 10.5 |
| β-Carotene | 0.3 |
| Echinenone | 1.4 |
| 3-Hydroxyechinenone | 0.9 |
| Canthaxanthin | 2.3 |
| Asteroidenone | 0.5 |
| Others | 4.4 |

To the resulting carotenoid composition, gum arabic, maltodextrin, tocopherol, medium-chain fatty acids and ascorbic acid were added and mixed together. The mixture was dissolved in water and then emulsified, followed by spray drying to obtain a carotenoid formulation (containing 1% by mass of astaxanthin, 0.14% by mass of adonirubin and 0.05% by mass of adonixanthin).

To 800 mg of the carotenoid formulation (containing 8 mg of astaxanthin, 1.12 mg of adonirubin and 0.4 mg of adonixanthin), a pH adjuster (896 mg), a sweetener (4,092 mg), a gelling agent (630 mg), a flavoring agent (200 mg) and water (33,382 mg) were added to prepare a carotenoid-containing food product. The thus prepared carotenoid-containing food product (40 g) was dispensed into four 10 g bags for jelly.

A carotenoid-free placebo was prepared (ingredients: an edible dye (2 mg), a pH adjuster (896 mg), a sweetener (4,092 mg), a gelling agent (630 mg), a flavoring agent (200 mg) and water (34,180 mg)).

### Inventive Example 1

Each subject in the subject group aged less than 55 years was allowed to take two bags of the carotenoid-containing food product twice a day, morning and evening after meals, i.e., four bags in total (the amount of carotenoids taken per day: 8 mg of astaxanthin, 1.12 mg of adonirubin and 0.4 mg of adonixanthin). The time of intake was not limited. Subjects who skipped breakfast were allowed to take the carotenoid-containing food product until 8:00 a.m. Also on the day of the test, all the subjects were allowed to take the carotenoid-containing food product. The carotenoid-containing food product was stored in a refrigerator.

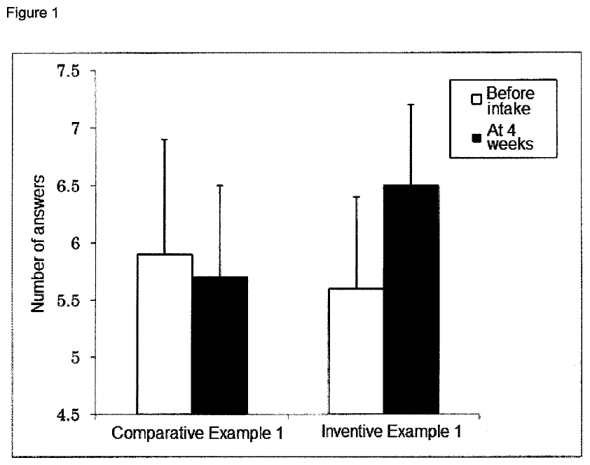

Word memory test was performed on the subject group at 4 weeks after initiation of intake. Table 4 shows changes in the measured values for all the items and their amounts of change compared with before intake. FIG. 1 shows the measured values for the “number of immediate free recall words+cued recall words” before intake and at 4 weeks after initiation of intake.

### Comparative Example 1

Each subject in the subject group aged less than 55 years was allowed to take the placebo in the same manner as shown in Inventive Example 1. Word memory test was performed on the subject group at 4 weeks after initiation of intake. Table 4 shows changes in the measured values for all the items and their amounts of change compared with before intake. FIG. 1 shows the measured values for the “number of immediate free recall words+cued recall words” before intake and at 4 weeks after initiation of intake.

<Comparison Between Inventive Example 1 and Comparative Example 1>

FIG. 1 shows the measured values for the “number of immediate free recall words+cued recall words” before intake and at 4 weeks after initiation of intake in Inventive Example 1 and the measured values for the “number of immediate free recall words+cued recall words” before intake and at 4 weeks after initiation of intake in Comparative Example 1.

At 4 weeks after initiation of administration, the amounts of changes in the following three items in Inventive Example 1, i.e., the “number of immediate free recall words+cued recall words,” the “number of words freely recalled after 5 min” and the “number of words freely recalled after 5 min+cued recall words” were significantly increased in comparison with Comparative Example 1 (bolded in the table, p<0.05; the “number of immediate free recall words+cued recall words”: Comparative Example 1—0.1±1.2 points, Inventive Example 1 0.9±1.1 points; the “number of words freely recalled after 5 min”: Comparative Example 1-0.4±1.8 points, Inventive Example 1 1.2±1.5 points; the “number of words freely recalled after 5 min +cued recall words”: Comparative Example 1-0.4±1.6 points, Inventive Example 1 0.9±1.4 points).

When compared between before and after intake, the amounts of changes in the “number of immediate free recall words+cued recall words” and the “number of words freely recalled after 5 min” in Inventive Example 1 were significantly increased (*p<0.05).

This result indicated that the subjects aged less than 55 years, i.e., the male and female subjects aged 45 to 54 years enhanced their word memory ability when allowed to take the carotenoid-containing food product. Namely, taking the carotenoid-containing food product from the early stage of aging was found to be useful in the prevention of reduction in the word memory ability or the maintenance or enhancement of the word memory ability.

TABLE 4
Word memory test in subjects aged less than 55 years (unit: the number of words)

| Item | Numerical item | | Before intake | At 4 weeks |
|---|---|---|---|---|
| Number of | Measured | Comp. Ex 1 | 4.7 ± 1.4 | 4.6 ± 1.1 |
| immediate free | value | Inv. Ex 1 | 4.6 ± 0.7 | 5.0 ± 0.9 |
| recall words | Amount of | Comp. Ex 1 | | −0.1 ± 1.8 |
| | change | Inv. Ex 1 | | 0.4 ± 1.0 |
| Number of | Measured | Comp. Ex 1 | 5.9 ± 1.0 | 5.7 ± 0.8 |
| immediate | value | Inv. Ex 1 | 5.6 ± 0.8 | 6.5 ± 0.7* |
| free recall | Amount of | Comp. Ex 1 | | −0.1 ± 1.2 |
| words + cued | change | Inv. Ex 1 | | 0.9 ± 1.1# |
| recall words | | | | |
| Number of | Measured | Comp. Ex 1 | 4.5 ± 1.3 | 4.1 ± 1.4 |
| words freely | value | Inv. Ex 1 | 4.4 ± 1.4 | 5.5 ± 0.9* |
| recalled after | Amount of | Comp. Ex 1 | | −0.4 ± 1.8 |
| 5 min | change | Inv. Ex 1 | | 1.2 ± 1.5# |
| Number of | Measured | Comp. Ex 1 | 5.4 ± 1.2 | 5.0 ± 1.0 |
| words freely | value | Inv. Ex 1 | 5.1 ± 0.9 | 6.0 ± 1.1 |
| recalled after | Amount of | Comp. Ex 1 | | −0.4 ± 1.6 |
| 5 min + cued | change | Inv. Ex 1 | | 0.9 ± 1.4# |
| recall words | | | | |

Comparative Example 1: n = 14,
Inventive Example 1: n = 11
Intra-group comparison with before intake
*p < 0.05,
**p < 0.01 (one-sample t-test)
Inter-group comparison with Comparative Example 1
#p < 0.05 (two-sample t-test)

### Inventive Example 2

Each subject in the subject group aged less than 55 years was allowed to take the carotenoid-containing food product in the same manner as shown in Inventive Example 1, followed by word recall tests. In Inventive Example 2, the intake period was set to 8 weeks. Table 5 shows changes in the measured values for all the items and their amounts of change compared with before intake.

### Comparative Example 2

Each subject in the subject group aged less than 55 years was allowed to take the carotenoid-free placebo in the same manner as shown in Comparative Example 1, followed by word recall tests. In Comparative Example 2, the intake period was set to 8 weeks. Table 5 shows changes in the measured values for all the items and their amounts of change compared with before intake.

### Comparison Between Inventive Example 2 and Comparative Example 2

In the word recall test for “vegetable names,” the amount of change at 4 weeks after initiation of intake was larger in Inventive Example 2 than in Comparative Example 2 (bolded in the table, p<0.1).

Moreover, although the inter-group difference was not significant at 8 weeks after initiation of intake, the amount of change in the number of words that could be recalled in Inventive Example 2 was larger (there were one or two words too many) than the amount of change in Comparative Example 2 in all the word recall tests for “vegetable names,” “words beginning with “a”” and “animal names.”

These results demonstrated that the subjects aged less than 55 years, i.e., the male and female subjects aged 45 to 54 years enhanced their word recall ability when allowed to take the carotenoid-containing food product for 4 weeks or longer. Namely, taking the carotenoid-containing food product from the early stage of aging was found to be useful in the prevention of reduction in the word recall ability or the maintenance or enhancement of the word recall ability.

TABLE 5
Word recall tests in subjects aged less than 55 years

| Item | Numerical item | | Before intake | At 4 weeks | At 8 weeks |
|---|---|---|---|---|---|
| Vegetable names | Measured | Comp. Ex 2 | 17.4 ± 3.5 | 17.1 ± 3.0 | 18.0 ± 3.4 |
| | value | Inv. Ex 2 | 15.9 ± 2.2 | 17.7 ± 2.8* | 17.6 ± 2.8** |
| | Amount of | Comp. Ex 2 | | −0.2 ± 2.8 | 0.6 ± 3.0 |
| | change | Inv. Ex 2 | | 1.8 ± 2.1† | 1.7 ± 1.7 |
| Words beginning | Measured | Comp. Ex 2 | 13.7 ± 3.7 | 13.2 ± 3.6 | 15.6 ± 5.4 |
| with “a” | value | Inv. Ex 2 | 14.1 ± 3.2 | 14.4 ± 4.8 | 17.5 ± 3.2** |
| | Amount of | Comp. Ex 2 | | −0.5 ± 3.0 | 1.9 ± 3.9 |
| | change | Inv. Ex 2 | | 0.3 ± 4.3 | 3.4 ± 2.9 |
| Animal names | Measured | Comp. Ex 2 | 19.6 ± 3.6 | 20.5 ± 3.3* | 21.1 ± 3.7* |
| | value | Inv. Ex 2 | 19.5 ± 4.5 | 20.4 ± 4.9 | 22.5 ± 3.8* |
| | Amount of | Comp. Ex 2 | | 0.9 ± 1.4 | 1.6 ± 2.2 |
| | change | Inv. Ex 2 | | 0.8 ± 4.8 | 2.9 ± 3.3 |

Comparative Example 2: n = 14,
Inventive Example 2: n = 11
Intra-group comparison with before intake
*p < 0.05,
**p < 0.01 (one-sample t-test)
Inter-group comparison with Comparative Example 2
†p < 0.1 (two-sample t-test)

### Inventive Example 3

Each subject in the subject group aged 55 years and over (55 to 64 years) was allowed to take the carotenoid-containing food product in the same manner as shown in Inventive Example 1, followed by Stroop test. Table 6 shows the results obtained for the total number of answers, Table 7 shows the results obtained for the number of correct answers, and Table 8 shows the results obtained for the number of wrong answers.

### Comparative Example 3

Each subject in the subject group aged 55 years and over (55 to 64 years) was allowed to take the placebo in the same manner as shown in Comparative Example 1, followed by Stroop test. Table 6 shows the results obtained for the total number of answers, Table 7 shows the results obtained for the number of correct answers, and Table 8 shows the results obtained for the number of wrong answers.

### Comparison Between Inventive Example 3 and Comparative Example 3

The amount of change in the total number of answers at 4 weeks in step 1, the amount of change in the number of correct answers at 4 and 8 weeks in step 1, and the amount of change in the number of correct answers at 4 weeks in step 3 were greatly increased in Inventive Example 3 when compared to Comparative Example 3 (p<0.1). As can be seen from the amount of change at 8 weeks in step 3, the number of wrong answers was greatly reduced in Inventive Example 3 when compared to Comparative Example 3 (p<0.1).

Namely, in the Stroop test, the carotenoid-containing food product group aged 55 years and over tended to show improvements in the total number of answers in step 1, the number of correct answers in steps 1 and 3, and the number of wrong answers in step 3.

This result indicated that the subjects aged 55 years and over, i.e., the male and female subjects aged 55 to 64 years enhanced their information processing ability when allowed to take the carotenoid-containing food product for 4 weeks or longer. Namely, taking the carotenoid-containing food product in and after the early stages of aging was found to be useful in the prevention or inhibition of reduction in the brain function, including the prevention of reduction in the information processing ability or the maintenance or maintenance of the information processing ability.

TABLE 6
Stroop test in subjects aged 55 years and over (total number of answers)

| Item | Numerical item | | Before intake | At 4 weeks | At 8 weeks |
|---|---|---|---|---|---|
| Step 1 | Measured | Comp. Ex 3 | 58.3 ± 6.4 | 57.9 ± 7.5 | 59.8 ± 8.8 |
| | value | Inv. Ex 3 | 56.4 ± 6.4 | 59.6 ± 7.6* | 61.0 ± 6.2** |
| | Amount of | Comp. Ex 3 | | −0.3 ± 5.8 | 1.5 ± 5.8 |
| | change | Inv. Ex 3 | | 3.2 ± 4.8† | 4.6 ± 4.2 |
| Step 2 | Measured | Comp. Ex 3 | 48.6 ± 5.3 | 50.3 ± 4.7 | 51.3 ± 7.4 |
| | value | Inv. Ex 3 | 48.6 ± 5.9 | 51.5 ± 6.3* | 52.9 ± 5.5** |
| | Amount of | Comp. Ex 3 | | 1.8 ± 3.9 | 2.8 ± 5.6 |
| | change | Inv. Ex 3 | | 2.9 ± 4.2 | 4.2 ± 4.8 |
| Step 3 | Measured | Comp. Ex 3 | 38.5 ± 3.4 | 38.9 ± 3.7 | 41.4 ± 4.1** |
| | value | Inv. Ex 3 | 39.2 ± 3.6 | 41.5 ± 4.4** | 41.9 ± 4.3** |
| | Amount of | Comp. Ex 3 | | 0.4 ± 3.1 | 2.9 ± 3.2 |
| | change | Inv. Ex 3 | | 2.4 ± 3.1 | 2.7 ± 3.5 |
| Step 4 | Measured | Comp. Ex 3 | 33.8 ± 3.8 | 35.0 ± 3.1 | 36.3 ± 4.2 |
| | value | Inv. Ex 3 | 33.5 ± 4.4 | 34.5 ± 4.0 | 35.5 ± 4.7 |
| | Amount of | Comp. Ex 3 | | 1.2 ± 2.4 | 2.4 ± 3.9 |
| | change | Inv. Ex 3 | | 1.0 ± 5.4 | 2.0 ± 5.3 |

All the data are expressed as mean ± standard deviation.
Comparative Example 3: n = 12,
Inventive Example 3: n = 17
Intra-group comparison with before intake
*p < 0.05,
**p < 0.01 (one-sample t-test)
Inter-group comparison with Comparative Example 3
†p < 0.1 (two-sample t-test)

TABLE 7
Stroop test in subjects aged 55 years and over (number of correct answers)

| Item | Numerical item | | Before intake | At 4 weeks | At 8 weeks |
|---|---|---|---|---|---|
| Step 1 | Measured | Comp. Ex 3 | 58.1 ± 6.2 | 57.6 ± 7.3 | 59.5 ± 8.7 |
| | value | Inv. Ex 3 | 56.3 ± 6.3 | 59.5 ± 7.5* | 61.0 ± 6.2** |
| | Amount of | Comp. Ex 3 | | −0.5 ± 5.6 | 1.4 ± 5.7 |
| | change | Inv. Ex 3 | | 3.2 ± 4.9† | 4.7 ± 4.1† |
| Step 2 | Measured | Comp. Ex 3 | 47.8 ± 6.0 | 49.6 ± 4.5 | 50.6 ± 7.8 |
| | value | Inv. Ex 3 | 48.1 ± 6.2 | 51.1 ± 6.3* | 52.7 ± 5.3** |
| | Amount of | Comp. Ex 3 | | 1.8 ± 4.7 | 2.8 ± 7.0 |
| | change | Inv. Ex 3 | | 3.0 ± 4.9 | 4.6 ± 5.0 |
| Step 3 | Measured | Comp. Ex 3 | 38.3 ± 3.5 | 38.8 ± 3.7 | 41.2 ± 4.2** |
| | value | Inv. Ex 3 | 38.8 ± 3.7 | 41.5 ± 4.3** | 41.8 ± 4.3** |
| | Amount of | Comp. Ex 3 | | 0.5 ± 3.1 | 2.8 ± 3.2 |
| | change | Inv. Ex 3 | | 2.6 ± 3.2† | 3.0 ± 3.6 |
| Step 4 | Measured | Comp. Ex 3 | 33.5 ± 4.1 | 34.8 ± 3.4 | 36.0 ± 4.4 |
| | value | Inv. Ex 3 | 33.4 ± 4.5 | 34.2 ± 4.1 | 35.4 ± 5.0 |
| | Amount of | Comp. Ex 3 | | 1.3 ± 2.7 | 2.5 ± 4.1 |
| | change | Inv. Ex 3 | | 0.8 ± 5.2 | 1.9 ± 5.6 |

All the data are expressed as mean ± standard deviation.
Comparative Example 3: n = 12,
Inventive Example 3: n = 17
Intra-group comparison with before intake
*p < 0.05,
**p < 0.01 (one-sample t-test)
Inter-group comparison with Comparative Example 3
†p < 0.1 (two-sample t-test)

TABLE 8
Stroop test in subjects aged 55 years
and over (number of wrong answers)

| Item | Numerical item | | Before intake | At 8 weeks |
|---|---|---|---|---|
| Step 1 | Measured | Comp. Ex 3 | 0.2 ± 0.4 | 0.3 ± 0.5 |
| | value | Inv. Ex 3 | 0.1 ± 0.5 | 0.0 ± 0.0 |
| | Amount of | Comp. Ex 3 | | 0.1 ± 0.5 |
| | change | Inv. Ex 3 | | −0.1 ± 0.5 |
| Step 2 | Measured | Comp. Ex 3 | 0.8 ± 1.5 | 0.8 ± 1.5 |
| | value | Inv. Ex 3 | 0.5 ± 0.9 | 0.2 ± 0.5 |
| | Amount of | Comp. Ex 3 | | 0.0 ± 2.3 |
| | change | Inv. Ex 3 | | −0.4 ± 1.2 |
| Step 3 | Measured | Comp. Ex 3 | 0.2 ± 0.4 | 0.3 ± 0.5 |
| | value | Inv. Ex 3 | 0.4 ± 0.6 | 0.1 ± 0.2 |
| | Amount of | Comp. Ex 3 | | 0.1 ± 0.5 |
| | change | Inv. Ex 3 | | −0.3 ± 0.6† |
| Step 4 | Measured | Comp. Ex 3 | 0.3 ± 0.5 | 0.3 ± 0.6 |
| | value | Inv. Ex 3 | 0.1 ± 0.2 | 0.1 ± 0.5 |
| | Amount of | Comp. Ex 3 | | −0.1 ± 0.8 |
| | change | Inv. Ex 3 | | 0.1 ± 0.6 |

Comparative Example 3: n = 12,
Inventive Example 3: n = 17
Inter-group comparison with Inventive Example 3
†p < 0.1 (two-sample t-test)

### Inventive Example 4

Analysis of word memory test results was performed on a group of subjects (45 to 54 years) showing a 0.100 μg/mL or higher increase in their blood astaxanthin (total) level, a 0.010 μg/mL or higher increase in their adonixanthin level and a 0.030 μg/mL or higher increase in their adonirubin level when compared with before intake of the carotenoid-containing food product. The results obtained are shown in Table 9.

### Inventive Example 5

Analysis of word memory test results was performed on a group of subjects (45 to 54 years) showing a 0.175 μg/mL or higher increase in their blood astaxanthin (total) level, a 0.020 μg/mL or higher increase in their adonixanthin level and a 0.030 μg/mL or higher increase in their adonirubin level when compared with before intake of the carotenoid-containing food product. The results obtained are shown in Table 9.

### Comparative Example 4

Analysis of word memory test results was performed on a group of placebo-receiving subjects (45 to 54 years). The results obtained are shown in Table 9.

### Comparison Between Inventive Examples 4 and 5 and Comparative Example 4

The means of the amounts of changes in all the items at 4 weeks and in all the items at 8 weeks in Inventive Examples 4 and 5 were larger than those of Comparative Example 4. Moreover, upon comparison between Inventive Example 4 and Inventive Example 5, Inventive Example 5 showed larger means of the amounts of changes in all but the “number of words freely recalled after 5 min” at 4 weeks. Inventive Example 4 showed an increased amount of change in the “number of words freely recalled after 5 min” at 4 weeks when compared to Comparative Example 4 (p<0.1).

In view of the foregoing, subjects with higher blood astaxanthin, adonixanthin and adonirubin levels were more likely to improve their word memory ability.

TABLE 9
Analysis on subjects with high serum astaxanthin (total) level

| | Numerical | | | | | |
|---|---|---|---|---|---|---|
| Item | item | n | | Before intake | At 4 weeks | At 8 weeks |
| Number of | Measured | 14 | Comp. Ex 4 | 4.7 ± 1.4 | 4.6 ± 1.1 | 5.1 ± 1.1 |
| immediate free recall | value | 7 | Inv. Ex 4 | 4.6 ± 0.8 | 4.6 ± 0.5 | 5.3 ± 1.3 |
| words | | 3 | Inv. Ex 5 | 4.7 ± 0.6 | 6.0 ± 1.0 | 6.0 ± 1.0* |
| | Amount of | 14 | Comp. Ex 4 | | −0.1 ± 1.8 | 0.4 ± 2.1 |
| | change | 7 | Inv. Ex 4 | | 0.0 ± 0.6 | 0.7 ± 1.3 |
| | | 3 | Inv. Ex 5 | | 1.3 ± 1.5 | 1.3 ± 1.2 |
| Number of | Measured | 14 | Comp. Ex 4 | 5.9 ± 1.0 | 5.7 ± 0.8 | 6.3 ± 1.0 |
| immediate free recall | value | 7 | Inv. Ex 4 | 5.7 ± 0.8 | 6.4 ± 0.8 | 6.3 ± 0.8 |
| words + cued recall words | | 3 | Inv. Ex 5 | 5.7 ± 1.2 | 6.7 ± 0.6 | 7.0 ± 0.0 |
| | Amount of | 14 | Comp. Ex 4 | | −0.1 ± 1.2 | 0.4 ± 1.4 |
| | change | 7 | Inv. Ex 4 | | 0.7 ± 1.0 | 0.6 ± 1.1 |
| | | 3 | Inv. Ex 5 | | 1.0 ± 1.7 | 1.3 ± 1.2 |
| Number of | Measured | 14 | Comp. Ex 4 | 4.5 ± 1.3 | 4.1 ± 1.4 | 5.4 ± 1.2* |
| words freely recalled | value | 7 | Inv. Ex 4 | 3.9 ± 1.5 | 5.1 ± 0.9 | 5.1 ± 1.1* |
| after 5 min | | 3 | Inv. Ex 5 | 5.0 ± 1.0 | 6.0 ± 0.0 | 6.7 ± 0.6* |
| | Amount of | 14 | Comp. Ex 4 | | −0.4 ± 1.8 | 0.9 ± 1.4 |
| | change | 7 | Inv. Ex 4 | | 1.3 ± 1.8† | 1.3 ± 1.3 |
| | | 3 | Inv. Ex 5 | | 1.0 ± 1.0 | 1.7 ± 0.6 |
| Number of | Measured | 14 | Comp. Ex 4 | 5.4 ± 1.2 | 5.0 ± 1.0 | 6.0 ± 1.2 |
| words freely recalled | value | 7 | Inv. Ex 4 | 4.9 ± 1.1 | 5.7 ± 1.3 | 6.0 ± 0.8* |
| after 5 min + cued recall words | | 3 | Inv. Ex 5 | 5.4 ± 0.6 | 6.3 ± 0.6 | 6.7 ± 0.6* |
| | Amount of | 14 | Comp. Ex 4 | | −0.4 ± 1.6 | 0.6 ± 1.5 |
| | change | 7 | Inv. Ex 4 | | 0.9 ± 1.8 | 1.1 ± 1.2 |
| | | 3 | Inv. Ex 5 | | 1.0 ± 1.0 | 1.3 ± 0.6 |

Intra-group comparison with before intake *p < 0.05 (one-sample t-test)
Inter-group comparison with Comparative Example 4 †p < 0.1 (two-sample t-test)

As shown above, in Inventive Examples 1 to 5 performed on healthy middle-aged and elderly male and female subjects aged 45 to 64 years, the composition or formulation of the present invention was found to be useful in the prevention of reduction in the word memory ability or the maintenance or enhancement of the word memory ability and also useful in the prevention of reduction in the word recall ability or the maintenance or enhancement of the word recall ability in subjects aged less than 55 years who were in the early stage of aging. Furthermore, the composition or formulation of the present invention was found to be useful in the prevention of reduction in the information processing ability or the maintenance or enhancement of the information processing ability in subjects aged 55 years and over who were in and after the early stages of aging.

The carotenoids used in the inventive examples of the present invention are derived from bacteria belonging to the genus Paracoccus, and include not only astaxanthin but also other carotenoids such as adonirubin and adonixanthin. On the other hand, carotenoids produced by Haematococcus pluvialis are mostly limited to astaxanthin, and it is known that when a carotenoid composition derived from Haematococcus pluvialis was examined for its improving effect on the brain function, there was no statistical significance between the placebo group and the group receiving the carotenoid composition (J Clin Biochem Nutr. 2012 September; 51(2): 102-107). The mechanism by which the carotenoid composition or formulation of the present invention exerts its improving effect on the brain function has not yet been identified exactly, but adonirubin and adonixanthin are deemed to be efficiently transferred into the blood of subjects when compared to astaxanthin, and then accumulated in or around the brain to exert their function.

### Reference Example

The subject group aged less than 55 years and the subject group aged 55 years and over were examined for their serum carotenoids. Table 10 shows changes in the measured values for all items and their amounts of change compared with before intake. In both the subject group aged less than 55 years and the subject group aged 55 years and over, the amounts of changes in all the items at 8 weeks were significantly increased in the inventive example group when compared to the comparative example group.

Serum astaxanthin in the group receiving the carotenoid-containing food product was found to contain its trans, 9-cis and 13-cis forms. Moreover, the group receiving the carotenoid-containing food product was found to contain adonixanthin in trans form and adonirubin in trans form in the serum.

TABLE 10
Blood carotenoids in the subject group aged less than 55 years

| Item | Numerical item | | Before intake | At 8 weeks |
|---|---|---|---|---|
| Astaxanthin | Measured | Comp. Ex 1 | 0.000 ± 0.000 | 0.000 ± 0.000 |
| (trans form) | value | Inv. Ex 1 | 0.000 ± 0.000 | 0.053 ± 0.013** |
| | Amount of | Comp. Ex 1 | | 0.000 ± 0.000 |
| | change | Inv. Ex 1 | | 0.053 ± 0.013## |
| Astaxanthin | Measured | Comp. Ex 1 | 0.000 ± 0.000 | 0.000 ± 0.000 |
| (9-cis form) | value | Inv. Ex 1 | 0.000 ± 0.000 | 0.027 ± 0.010** |
| | Amount of | Comp. Ex 1 | | 0.000 ± 0.000 |
| | change | Inv. Ex 1 | | 0.027 ± 0.010## |
| Astaxanthin | Measured | Comp. Ex 1 | 0.000 ± 0.000 | 0.000 ± 0.000 |
| (13-cis form) | value | Inv. Ex 1 | 0.000 ± 0.000 | 0.074 ± 0.026** |
| | Amount of | Comp. Ex 1 | | 0.000 ± 0.000 |
| | change | Inv. Ex 1 | | 0.074 ± 0.026## |
| Astaxanthin | Measured | Comp. Ex 1 | 0.000 ± 0.000 | 0.000 ± 0.000 |
| (total) | value | Inv. Ex 1 | 0.000 ± 0.000 | 0.154 ± 0.046** |
| | Amount of | Comp. Ex 1 | | 0.000 ± 0.000 |
| | change | Inv. Ex 1 | | 0.154 ± 0.046## |
| Adonixanthin | Measured | Comp. Ex 1 | 0.000 ± 0.000 | 0.000 ± 0.000 |
| (trans form) | value | Inv. Ex 1 | 0.000 ± 0.000 | 0.015 ± 0.005** |
| | Amount of | Comp. Ex 1 | | 0.000 ± 0.000 |
| | change | Inv. Ex 1 | | 0.015 ± 0.005## |
| Adonirubin | Measured | Comp. Ex 1 | 0.000 ± 0.000 | 0.000 ± 0.000 |
| (trans form) | value | Inv. Ex 1 | 0.000 ± 0.000 | 0.037 ± 0.012** |
| | Amount of | Comp. Ex 1 | | 0.000 ± 0.000 |
| | change | Inv. Ex 1 | | 0.037 ± 0.012## |

All the data are expressed as mean ± standard deviation (unit: μg/mL).
Comparative Example 1: n = 14,
Inventive Example 1: n = 11
Intra-group comparison with before intake
**p < 0.01 (one-sample t-test)
Inter-group comparison with Comparative Example 1
##p < 0.01 (two-sample t-test)

TABLE 11
Blood carotenoids in the subject group aged 55 years and over

| Item | Numerical item | | Before intake | At 8 weeks |
|---|---|---|---|---|
| Astaxanthin | Measured | Comp. Ex 3 | 0.000 ± 0.000 | 0.000 ± 0.000 |
| (trans form) | value | Inv. Ex 3 | 0.000 ± 0.000 | 0.064 ± 0.027** |
| | Amount of | Comp. Ex 3 | | 0.000 ± 0.000 |
| | change | Inv. Ex 3 | | 0.064 ± 0.027## |
| Astaxanthin | Measured | Comp. Ex 3 | 0.000 ± 0.000 | 0.000 ± 0.000 |
| (9-cis form) | value | Inv. Ex 3 | 0.000 ± 0.000 | 0.031 ± 0.013** |
| | Amount of | Comp. Ex 3 | | 0.000 ± 0.000 |
| | change | Inv. Ex 3 | | 0.031 ± 0.013## |
| Astaxanthin | Measured | Comp. Ex 3 | 0.000 ± 0.000 | 0.000 ± 0.000 |
| (13-cis form) | value | Inv. Ex 3 | 0.000 ± 0.000 | 0.091 ± 0.031** |
| | Amount of | Comp. Ex 3 | | 0.000 ± 0.000 |
| | change | Inv. Ex 3 | | 0.091 ± 0.031## |
| Astaxanthin | Measured | Comp. Ex 3 | 0.000 ± 0.000 | 0.000 ± 0.000 |
| (total) | value | Inv. Ex 3 | 0.000 ± 0.000 | 0.186 ± 0.063** |
| | Amount of | Comp. Ex 3 | | 0.000 ± 0.000 |
| | change | Inv. Ex 3 | | 0.186 ± 0.063## |
| Adonixanthin | Measured | Comp. Ex 3 | 0.000 ± 0.000 | 0.000 ± 0.000 |
| (trans form) | value | Inv. Ex 3 | 0.000 ± 0.000 | 0.017 ± 0.007** |
| | Amount of | Comp. Ex 3 | | 0.000 ± 0.000 |
| | change | Inv. Ex 3 | | 0.017 ± 0.007## |
| Adonirubin | Measured | Comp. Ex 3 | 0.000 ± 0.000 | 0.000 ± 0.000 |
| (trans form) | value | Inv. Ex 3 | 0.000 ± 0.000 | 0.045 ± 0.014** |
| | Amount of | Comp. Ex 3 | | 0.000 ± 0.000 |
| | change | Inv. Ex 3 | | 0.045 ± 0.014## |

All the data are expressed as mean ± standard deviation (unit: μg/mL).
Comparative Example 3: n = 12,
Inventive Example 3: n = 17
Intra-group comparison with before intake
**p < 0.01 (one-sample t-test)
Inter-group comparison with Comparative Example 3
##p < 0.01 (two-sample t-test)

### INDUSTRIAL APPLICABILITY

The present invention provides a prophylactic agent for brain hypofunction or an inhibitory agent for brain hypofunction. Moreover, in a preferred embodiment, the present invention provides a prophylactic agent for aging-induced brain hypofunction or an inhibitory agent for aging-induced brain hypofunction.

1. An inhibitory or prophylactic agent for brain hypofunction, which comprises a carotenoid composition containing astaxanthin, adonirubin and adonixanthin.

2. The agent according to claim 1, wherein the carotenoid composition contains 45% to 80% by mass of astaxanthin, 4% to 22% by mass of adonirubin and 4% to 20% by mass of adonixanthin, on the basis of the total mass of the composition.

3. An inhibitory or prophylactic agent for brain hypofunction, which comprises a carotenoid formulation containing astaxanthin, adonirubin and adonixanthin.

4. The agent according to claim 3, wherein the carotenoid formulation contains 0.5% to 20% by mass of astaxanthin, 0.01% to 4% by mass of adonirubin and 0.01% to 4% by mass of adonixanthin, on the basis of the total mass of the formulation.

5. The agent according to claim 1, wherein the astaxanthin has no ester structure.

6. The agent according to claim 1, wherein the adonirubin has no ester structure.

7. The agent according to claim 1, wherein the adonixanthin has no ester structure.

8. The agent according to claim 1, wherein the brain function is verbal memory ability.

9. The agent according to claim 1, wherein the brain function is information processing ability.

10. The agent according to claim 1, wherein the brain hypofunction is aging-induced brain hypofunction.

11. A pharmaceutical composition, a food composition or a drinkable composition, which contains the agent according to claim 1.

12. A method for inhibiting or preventing brain hypofunction, which comprises administering a carotenoid composition or a carotenoid formulation, each containing astaxanthin, adonirubin and adonixanthin, to a subject.

13. The method according to claim 12, wherein the carotenoid composition contains 45% to 80% by mass of astaxanthin, 4% to 22% by mass of adonirubin and 4% to 20% by mass of adonixanthin, on the basis of the total mass of the composition.

14. The method according to claim 12, wherein the carotenoid formulation contains 0.5% to 20% by mass of astaxanthin, 0.01% to 4% by mass of adonirubin and 0.01% to 4% by mass of adonixanthin, on the basis of the total mass of the formulation.

15. The method according to claim 12, wherein the brain function is verbal memory ability or information processing ability.

16. The method according to claim 12, wherein the brain hypofunction is aging-induced brain hypofunction.
